(19) United States
(12) Patent Application Publication
(10) Pub. No.: US 2002/0009491 A1
(43) Pub. Date: Jan. 24, 2002

(54) Compositions and methods for enhancing drug delivery across biological membranes and tissues

(76) Inventors: Jonathan B. Rothbard, Cupertino, CA (US); Paul A. Wender, Menlo Park, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/779,693

(22) Filed: Feb. 7, 2001

Related U.S. Application Data
(60) Provisional application No. 60/182,166, filed on Feb. 14, 2000.

(51) Int. Cl. A61K 9/14

(52) U.S. Cl. 424/484; 424/486

(57) ABSTRACT

This invention provides compositions and methods for enhancing delivery of drugs and other agents across a biological barrier, including epithelial tissues such as the skin, gastrointestinal tract, pulmonary epithelium, and the like. The compositions and methods are also useful for delivery across endothelial tissues, including the blood brain barrier. The compositions and methods employ a delivery enhancing transporter that has sufficient guanidino or amidino sidechain moieties to enhance delivery of a compound across one or more layers of the tissue, compared to the non-conjugated compound. The delivery-enhancing polymers include, for example, poly-arginine molecules that are preferably between about 6 and 50 residues in length.

### BACKGROUND OF THE INVENTION

1. Field of the Invention

This invention pertains to the field of compositions and methods that enhance the delivery of drugs and other compounds across biological membranes and tissues, including, for example, cell membranes, mitochondrial membranes, and dermal and epithelial membranes.

2. Background

Cell membranes and biological tissues often present a formidable barrier between a therapeutic agent and its desired target site. For example, a therapeutic agent may be hydrophilic and freely soluble in the aqueous compartments of the body, but cannot penetrate the lipid layers that surround cells. Similarly, a therapeutic agent may be so insoluble in an aqueous environment that it is difficult to formulate for suitable administration. As a result, while advances in screening technologies, biotechnology and the like have made a significant impact on the number of potentially valuable therapeutic agents, considerations of appropriate drug delivery have often hindered their medical utility.

One approach to this problem has involved the use of transporter molecules (e.g., liposomes or lipid particles) to escort compounds across biological membranes. Others have used high molecular weight polymers of lysine for increasing transport of various molecules across cellular membranes, with very high molecular weights being preferred (see, Ryser et al. (1979)). Although the authors contemplated polymers of other positively charged residues such as ornithine and arginine, operativity of such polymers was not shown.

Frankel et al. (1991) reported that conjugating selected molecules to the tat protein of HIV can increase cellular uptake of those molecules. However, use of the tat protein has certain disadvantages, including unfavorable aggregation and insolubility properties.

Barsoum et al. (1994) and Fawell et al. (1994) proposed using shorter fragments of the tat protein containing the tat basic region (residues 49-57 having the sequence RKKRRQRRR. Barsoum et al. noted that moderately long polyarginine polymers (MW 5000-15000 daltons) failed to enable transport of β-galactosidase across cell membranes (e.g., Barsoum on page 3), contrary to the suggestion of Ryser et al. (supra).

Transdermal or transmucosal drug delivery, while an attractive route of drug delivery, presents other considerations and hurdles (see U.S. Ser. No. 60/150,510, filed Aug. 24, 1999).

Among the methods proposed to enhance transdermal transport of drugs are chemical enhancers (Burnette, R. R. In Developmental Issues and Research Initiatives; Hadgraft J., Ed., Marcel Dekker: 1989; pp. 247-288) and iontophoresis. However, in spite of the more than thirty years of research that has gone into delivery of drugs across the skin in particular, fewer than a dozen drugs are now available for transdermal administration in, for example, skin patches.

Yet another barrier to certain drugs is the blood-brain barrier. The brain capillaries that make up the blood-brain barrier are composed of endothelial cells that form tight junctions between themselves (Goldstein et al., Scientific American 255:74-83 (1986); Pardridge, W. M., Endocrin. Rev. 7: 314-330 (1986)). The endothelial cells and the tight intercellular junctions that join the cells form a barrier against the passive movement of many molecules from the blood to the brain.

Thus, a need exists for improved compositions and methods for enhancing delivery of compounds, including drugs, to the surface of cell membranes and certain tissues, and across cell membranes as well as epithelial tissues and endothelial tissues such as the skin and the blood-brain barrier. The present invention fulfills this and other needs.

### SUMMARY OF THE INVENTION

The present invention provides compositions and methods for enhancing delivery of a compound to the surface of, into, or across a biological membrane, including a cell membrane, plasma membrane, nuclear membrane, or into or across one or more layers of an animal epithelial or endothelial tissue. The methods involve contacting the membrane or tissue with a composition that includes the compound in association with at least one delivery-enhancing transporter. The delivery-enhancing transporters, described herein, have sufficient guanidino or amidino moieties to provide association with a cell surface which can lead to increased delivery of the compound into and across one or more membranes or intact epithelial or endothelial tissue layers compared to delivery of the compound in the absence of the delivery-enhancing transporter. Typically, the delivery-enhancing transporters have from 6 to 50 guanidino or amidino moieties, and more preferably between 7 and 15 guanidino moieties.

The compositions described herein are compositions in which a therapeutic agent or a suitable derivative is combined with a delivery-enhancing transporter to form a complex in which the components are not joined covalently. Typically, the complex is the result of ion pairing.

The compositions and methods of the invention are useful for delivering drugs, diagnostic agents, and other compounds of interest across cell membranes, nuclear membranes, plasma membranes, and epithelial tissues such as the skin and mucous membranes. Delivery across the blood-brain barrier can also be enhanced by the compositions of the invention. The methods and compositions of the invention can be used not only to deliver the compounds to the particular site of administration, but also provide systemic delivery.

In one embodiment, the invention provides a method for treating a skin condition. The methods involve contacting an area of skin affected by the skin condition with a composition comprising a therapeutic compound and a delivery-enhancing transporter. The delivery-enhancing transporter includes sufficient guanidino or amidino moieties to carry the compound across a biological membrane at a rate that is greater than the trans-membrane transport rate of the biologically active agent in non-combined form.

Additional embodiments of the invention provide transdermal drug formulations. These formulations include a therapeutically effective amount of a therapeutic agent, a delivery-enhancing polymer that includes sufficient guanidino or amidino sidechain moieties to increase delivery of the conjugate across one or more layers of an animal epithelial tissue compared to the trans-epithelial tissue delivery of the biologically active agent in non-conjugated form; and a vehicle suited to transdermal drug administration.

### BRIEF DESCRIPTION OF THE FIGURES

FIG. 1. This figure illustrates a modified taxane composition in which the C'2 group of taxol has been derivatized to include a phosphate residue which forms a complex with a heptamer of arginine residues.

FIG. 2. This figure illustrates a non-covalent composition formed between fluorescein and a nonamer of arginine residues.

FIG. 3. Staining of lymphocytes using different salts between fluorescein and nonamers of arginine. Human lymphocytes (Jurkat) were incubated with varying concentrations of each of the fluorescein-polyarginne salts for five minutes in PBS/2% fetal calf serum at room temperature. The cells were washed with PBS, exposed to 0.1% propidium iodide and analyzed by flow cytometry. The mean fluorescence of 10⁴ cells is shown.

FIG. 4. Micrographs of lymphocyte stained with a salt of fluorescein and a nonamer of L-arginine. Fluorescent and transmission views of the same field demonstrate that all cells were highly stained. The human t cell line, Jurkat, was exposed to 50 uM solution of the 1:1 salt of fluorescein and R9 for five minutes at room temperature. The cells were washed, and placed on a coverslip and analyzed using a fluorescent microscope.

FIG. 5. Cytotoxicity assay demonstrating that the taxol-heptaarginine salt was equally potent at killing lymphocytes as taxol dissolved in dimethyl sulfoxide. Cells were incubated with varying concentrations of either taxol dissolved in DMSO or the 1:1 taxol heptaarginine salt dissolved in PBS for 3 days at 37° C. At the end of this period, cells were exposed to 0.05 M MTT in PBS, incubated for one hour, spun, and incubated with acidic propanol for 2 hours. At the end of this incubation the optical density at 650 nm was measured, and the percentage of dead cells was calculated.

### DETAILED DESCRIPTION

Definitions

As used herein, the term “biological barrier” refers to a physiological barrier to the delivery of a drug to its intended target site and includes, for example, those barriers defined in more detail below as “biological membranes,” “epithelial tissue,” or “endothelial tissue.”

The term “biological membrane” as used herein refers to a lipid-containing barrier which separates cells or groups of cells from the extracellular space. Biological membranes include, but are not limited to, plasma membranes, cell walls, intracellular organelle membranes, such as the mitochondrial membrane, nuclear membranes, and the like.

An “epithelial tissue” is the basic tissue that covers surface areas of the surface, spaces, and cavities of the body. Epithelial tissues are composed primarily of epithelial cells that are attached to one another and rest on an extracellular matrix (basement membrane) that is typically produced by the cells. Epithelial tissues include three general types based on cell shape: squamous, cuboidal, and columnar epithelium. Squamous epithelium, which lines lungs and blood vessels, is made up of flat cells. Cuboidal epithelium lines kidney tubules and is composed of cube shaped cells, while columnar epithelium cells line the digestive tract and have a columnar appearance. Epithelial tissues can also be classified based on the number of cell layers in the tissue. For example, a simple epithelial tissue is composed of a single layer of cells, each of which sits on the basement membrane. A “stratified” epithelial tissue is composed of several cells stacked upon one another; not all cells contact the basement membrane. A “pseudostratified” epithelial tissue has cells that, although all contact the basement membrane, appear to be stratified because the nuclei are at various levels.

“Biologically active agent” or “biologically active substance” refers to a chemical substance, such as a small molecule, macromolecule, or metal ion, that causes an observable change in the structure, function, or composition of a cell upon uptake by the cell. Observable changes include increased or decreased expression of one or more mRNAs, increased or decreased expression of one or more proteins, phosphorylation of a protein or other cell component, inhibition or activation of an enzyme, inhibition or activation of binding between members of a binding pair, an increased or decreased rate of synthesis of a metabolite, increased or decreased cell proliferation, and the like. Included within this definition are “therapeutic agents”, “therapeutic compositions”, and “therapeutic substances” which refer, without limitation, to any composition that can be used to the benefit of a mammalian species. Such agents may take the form of ions, small organic molecules, peptides, proteins or polypeptides, and oligosaccharides, for example.

The term “transmembrane concentration” refers to the concentration of a compound present on the side of a membrane that is opposite or “trans” to the side of the membrane to which a particular composition has been added. For example, when a compound is added to the extracellular fluid of a cell, the amount of the compound measured subsequently inside the cell is the transmembrane concentration of the compound.

The term “trans-epithelial” delivery or administration refers to the delivery or administration of agents by permeation through one or more layers of a body surface or tissue, such as intact skin or a mucous membrane, by topical administration. Thus, the term is intended to include both transdermal (e.g., percutaneous adsorption) and transmucosal administration. Delivery can be to a deeper layer of the tissue, for example, and/or delivery to the bloodstream.

“Delivery enhancement”, “penetration enhancement” or “permeation enhancement” as used herein relates to an increase in amount and/or rate of delivery of a compound that is delivered into or across a biological membrane or into and across one or more layers of an epithelial or endothelial tissue. An enhancement of delivery can be observed by measuring the rate and/or amount of the compound that passes through one or more layers of animal or human skin, or other tissue or cell membrane. Delivery enhancement also can involve an increase in the depth into the tissue to which the compound is delivered, and/or the extent of delivery to one or more cell types of the epithelial or other tissue (e.g., increased delivery to fibroblasts, immune cells, and endothelial cells of the skin or other tissue). Such measurements are readily obtained by, for example, using a diffusion cell apparatus as described in U.S. Pat. No. 5,891,462.

The amount or rate of delivery of an agent across and/or into skin or other epithelial or endothelial membrane is sometimes quantitated in terms of the amount of compound passing through a predetermined area of skin, membrane or other tissue, which is a defined area of intact unbroken living skin or mucosal tissue. That area will usually be in the range of about 5 cm² to about 100 cm², more usually in the range of about 10 cm² to about 100 cm², still more usually in the range of about 20 cm² to about 60 cm².

The term “trans-barrier concentration” or “trans-tissue concentration” refers to the concentration of a compound present on the side of one or more layers of an epithelial or endothelial barrier tissue that is opposite or “trans” to the side of the tissue to which a particular composition has been added. For example, when a compound is applied to the skin, the amount of the compound measured subsequently across one or more layers of the skin is the trans-barrier concentration of the compound.

The terms “guanidyl,” “guanidinyl” and “guanidino” are used interchangeably to refer to a moiety having the formula —NHC(═NH)NH₂ (unprotonated form). As an example, arginine contains a guanidyl (guanidino) moiety, and is also referred to as 2-amino-5-guanidinovaleric acid or α-amino-δ-guanidinovaleric acid. “Guanidium” refers to the positively charged conjugate acid form. The term “guanidino moiety” includes, for example, guanidine, guanidinium, guanidine derivatives such as (RNHC(NH)NHR′), monosubstituted guanidines, monoguanides, biguanides, biguanide derivatives such as (RNHC(NH)NHC(NH)NHR′), and the like. In addition, the term “guanidino moiety” encompasses any one or more of a guanide alone or a combination of different guanides.

“Amidinyl” and “amidino” refer to a moiety having the formula —C(═NH)(NH₂). “Amidinium” refers to the positively charged conjugate acid form.

The term “macromolecule” as used herein refers to large molecules (MW greater than 1000 daltons) exemplified by, but not limited to, peptides and proteins, of biological or synthetic origin.

“Small organic molecule” refers to a carbon-containing agent having a molecular weight (MW) of less than or equal to 1000 daltons.

The term “polymer” refers to a linear chain of two or more identical or non-identical subunits joined by covalent bonds. A peptide is an example of a polymer that can be composed of identical or non-identical amino acid subunits that are joined by peptide linkages.

The term “peptide” as used herein refers to a compound made up of a single chain of D- or L- amino acids or a mixture of D- and L-amino acids joined by peptide bonds. Generally, peptides contain at least two amino acid residues and are less than about 50 amino acids in length. D-amino acids are represented herein by a lower-case one-letter amino acid symbol (e.g., r for D-arginine), whereas L-amino acids are represented by an upper case one-letter amino acid symbol (e.g., R for L-arginine). Homopolymer peptides are represented by a one-letter amino acid symbol followed by the number of consecutive occurrences of that amino acid in the peptide- (e.g., R7 represents a heptamer that consists of L-arginine residues).

The term “protein” as used herein refers to a compound that is composed of linearly arranged amino acids linked by peptide bonds, but in contrast to peptides, has a well-defined conformation. Proteins, as opposed to peptides, generally consist of chains of 50 or more amino acids.

“Polypeptide” as used herein refers to a polymer of at least two amino acid residues and which contains one or more peptide bonds. “Polypeptide” encompasses peptides and proteins, regardless of whether the polypeptide has a well-defined conformation.

### DESCRIPTION OF THE EMBODIMENTS

The present invention provides compositions and methods that enhance the transfer of compounds, including drugs and other biologically active compounds, to the surface of, into, or across a biological membrane or one or more layers of an animal epithelial or endothelial tissue. The methods typically involve contacting the tissue or membrane with a composition that includes the compound of interest in combination with at least one delivery-enhancing transporter. The delivery enhancing transporters provided by the invention are molecules that include sufficient guanidino or amidino moieties to increase delivery of the compound to the surface of, into or across a biological membrane or one or more intact epithelial and endothelial tissue layers. The methods and compositions are useful for trans-epithelial and trans-endothelial delivery of drugs and other biologically active molecules, and also for delivery of imaging and diagnostic molecules. The methods and compositions of the invention are particularly useful for delivery of compounds that require trans-epithelial or trans-endothelial transport to exhibit their biological effects, and that by themselves (without a delivery-enhancing transporter), are unable, or only poorly able, to cross such tissues and thus exhibit biological activity.

The compositions and methods of the invention provide significant advantages over previously available compositions and methods for delivering biological agents or for obtaining trans-epithelial and trans-endothelial tissue delivery of compounds of interest. In particular, the delivery-enhancing transporters make possible the delivery of drugs and other biological agents across tissues that were previously impenetrable to the drug or agent, or in some instances were poorly soluble in pharmaceutical carriers. For example, while delivery of drugs across skin was previously nearly impossible for all but a few compounds, the methods of the invention can deliver compounds not only into cells of a first layer of an epithelial tissue such as skin, but also across one or more layers of the skin. The blood brain barrier is also resistant to transport of drugs and other diagnostic and therapeutic reagents; however, the methods and compositions of the invention provide means to obtain such transport.

The delivery-enhancing transporters used and described herein increase delivery of the associated compound or agent into and across one or more intact epithelial or endothelial tissue layers compared to delivery of the compound in the absence of the delivery-enhancing transporter. The delivery-enhancing transporters can, in some embodiments, increase delivery of the associated compound or agent significantly over that obtained using the tat protein of HIV-1 (Frankel et al. (1991) PCT Pub. No. WO 91/09958). Delivery is also increased significantly over the use of shorter fragments of the tat protein containing the tat basic region (residues 49-57 having the sequence RKKRRQRRR) (Barsoum et al. (1994) WO 94/04686 and Fawell et al. (1994) Proc. Nat'l. Acad. Sci. USA 91: 664-668). Preferably, delivery obtained using the transporters of the invention is increased more than 2-fold, and still more preferably six-fold, over that obtained with tat residues 49-57.

Similarly, the delivery-enhancing transporters described herein can provide increased delivery compared to a 16 amino acid peptide-cholesterol conjugate derived from the Antennapedia homeodomain that is rapidly internalized by cultured neurons (Brugidou et al. (1995) Biochem. Biophys. Res. Commun. 214: 685-93). This region, residues 43-58 at minimum, has the amino acid sequence RQIKIWFQNRRMKWKK. The Herpes simplex protein VP22, like tat and the Antennapedia domain, was previously known to enhance transport into cells, but was not known to enhance transport into and across endothelial and epithelial membranes (Elliot and O'Hare (1997) Cell 88: 223-33; Dilber et al. (1999) Gene Ther. 6: 12-21; Phelan et al. (1998) Nat. Biotechnol. 16: 440-3). In presently preferred embodiments, the delivery-enhancing transporters provide significantly increased delivery compared to the Antennapedia homeodomain and to the VP22 protein.

### Compositions of Delivery-Enhancing Transporters and Biologically Active Agents

In one group of embodiments, the present invention provides a composition of a delivery-enhancing transporter and a biologically active agent. The composition is a non-covalent combination of the delivery-enhancing transporter and the biologically active agent. Rather than a covalent composition, the components are held in an ionic association, typically viewed as an ion pair. Despite the term “ion pair,” the invention will, in some embodiments, include compositions of one or more biologically active agents in association with one delivery-enhancing transporter.

Each of the composition components will posses an ionic charge at physiologic pH. More particularly, the transporter will be positively charged and the biologically active agent will be negatively charged. In some embodiments, the biologically active agent is a derivative of a neutral therapeutic agent which has been modified to include an acidic (or other negatively charged) group which is cleavable in vivo.

a. Delivery-Enhancing Transporters

The delivery-enhancing transporter components used in the compositions and methods of the invention are molecules that have sufficient guanidino and/or amidino moieties to increase delivery of a compound or agent with which the delivery-enhancing transporter is combined. The increased delivery of the compound or agent can be across one or more layers of an epithelial tissue (e.g., skin or mucous membrane) or an endothelial tissue (e.g., the blood-brain barrier) or a cell membrane. The delivery-enhancing transporters typically include 6 to 50 guanidino and/or amidino moieties, more preferably between 7 and 15 such moieties.

Illustrative of the delivery enhancing transporters are poly-Arg transporters consisting of heptamers, octamers, nonamers and the like of arginine. Similarly, polymers of homoarginine are useful as well as peptides comprising arginine residues in addition to other amino acid residues which can provide a particular structural feature to the transporter. For example, a decamer of arginine residues can be interrupted by one or more proline residues which are known to induce a β-turn conformation in peptides. In this manner, a transporter can be designed to potentially surround and further protect (prior to delivery) a therapeutic agent or other biological agent.

Other amino acids can be used in the transporters described herein, such as the commonly encountered and naturally occurring D- and L-amino acids (e.g., Gly, Leu, Val, Asp, Pro, Met, Trp, Phe, Ala and the like) as well as aminocaproic acid, sarcosine, phenylglycine, citrulline, aminoisobutyric acid, norleucine, norvaline, homoproline, aminobutyric acid, β-alanine, and the like.

In addition to the embodiments above, the delivery enhancing transporters will, in a broader sense, encompass essentially any poly guanidino, poly amidino or mixed poly guanidino/poly amidino-containing vehicle, wherein the guanidino and amidino moieties are sufficiently spaced to form a complex with a biological agent or therapeutic agent and further interact with the biological barrier to enhance the delivery of the agent into or across the biological barrier.

The transporters can be a linear configuration of guanidino and/or amidino groups on a backbone, a branched configuration (using, for example a lysine, aspartic acid or glutamic acid residue to form a branch in a peptide configuration), or a cyclic configuration.

Guanidino and/or Amidino Moieties

The delivery-enhancing transporters of the invention include guanidino and/or amidino moieties, which are involved in enhancing the transport of a complexed agent to the surface of, into or across a biological membrane. In some embodiments, the delivery-enhancing transporters are composed of linked subunits, at least some of which include a guanidino and/or amidino moiety. Examples of suitable subunits having guanidino and/or amidino moieties are described below.

Amino Acids

In some embodiments, the delivery-enhancing transporters are composed of D or L amino acid residues. Use of naturally occurring L-amino acid residues in the delivery-enhancing transporters has the advantage that break-down products should be relatively non-toxic to the cell or organism. Preferred amino acid subunits are arginine (α-amino-δ-guanidinovaleric acid), and α-amino-ε-amidino-hexanoic acid (isosteric amidino analog). The guanidinium group in arginine has a pKa of about 12.5.

More generally, it is preferred that each subunit contains a highly basic sidechain moiety which (i) has a pKa of greater than 11, more preferably 12.5 or greater, and (ii) contains, in its protonated state, at least two geminal amino groups (NH₂) which share a resonance-stabilized positive charge, which gives the moiety a bidentate character.

Other amino acids, such as α-amino-β-guanidino-propionic acid, α-amino-γ-guanidino-butyric acid, or α-amino-ε-guanidino-caproic acid can also be used (containing 2, 3 or 5 linker atoms, respectively, between the backbone chain and the central guanidinium carbon).

D-amino acids can also be used in the delivery enhancing transporters. Compositions containing exclusively D-amino acids have the advantage of decreased enzymatic degradation. However, they can also remain largely intact within the target cell. Such stability is generally not problematic if the agent is biologically active when the polymer is still attached.

Other Subunits

As noted above, subunits other than amino acids may also be selected for use in forming transport polymers. Such subunits may include, but are not limited to hydroxy amino acids, N-methyl-amino acids, amino aldehydes, and the like, which result in polymers with reduced peptide bonds. Other subunit types can be used, depending on the nature of the selected backbone, as discussed below.

Backbones

The guanidino and/or amidino moieties that are included in the delivery-enhancing transporters are generally attached to a linear backbone. The backbone can comprise heteroatoms selected from carbon, nitrogen, oxygen, sulfur, and phosphorus, with the majority of backbone chain atoms usually consisting of carbon. To the backbone are attached a plurality of sidechain moieties that include a terminal guanidino or amidino group. Although the spacing between adjacent sidechain moieties will usually be consistent, the delivery-enhancing transporters used in the invention can also include variable spacing between sidechain moieties along the backbone.

The guanidino or amidino moieties extend away from the backbone by virtue of being linked to the backbone by the sidechain linker. The sidechain atoms are preferably provided as methylene carbon atoms, although one or more other atoms such as oxygen, sulfur, or nitrogen can also be present. For example, an alkylene linker that attaches a guanidino moiety to a peptide-like backbone can be shown as:

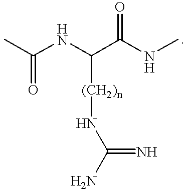

In this formula, n is preferably at least 2, and is preferably from about 2 to about 7. In some embodiments, n is 3, in which the sidechain is that of arginine. In presently preferred embodiments, n is from about 4 to about 6, most preferably n is 5 or 6. Although the exemplified formula is shown as being attached to a peptide backbone (i.e., a repeating amide to which the sidechain is attached to the carbon atom that is α to the carbonyl group), non-peptide backbones are also suitable, as discussed in more detail herein.

A variety of backbone types can be used to order and position the sidechain guanidino and/or amidino moieties, such as alkyl backbone moieties joined by thioethers or sulfonyl groups, hydroxy acid esters (equivalent to replacing amide linkages with ester linkages), replacing the α carbon of an α-amino acid with nitrogen to form an aza analog, alkyl backbone moieties joined by carbamate groups, polyethyleneimines (PEIs), and amino aldehydes, which result in polymers composed of secondary amines.

A more detailed backbone list includes N-substituted amide (CONR replaces CONH linkages), esters (CO₂), keto-methylene (COCH₂) reduced or methyleneamino (CH₂NH), thioamide (CSNH), phosphinate (PO₂RCH₂), phosphonamidate and phosphonamidate ester (PO₂RNH), retropeptide (NHCO), trans-alkene (CR═CH), fluoroalkene (CF═CH), dimethylene (CH₂CH₂), thioether (CH₂S), hydroxyethylene (CH(OH)CH₂), methyleneoxy (CH₂O), tetrazole (CN₄), retrothioamide (NHCS), retroreduced (NHCH₂), sulfonamido (SO₂NH), methylenesulfonamido (CHRSO₂NH), retrosulfonamide (NHSO₂), and peptoids (N-substituted amides), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from α-amino acids.

Peptoid backbones can also be used (e.g., Kessler (1993) Angew. Chem. Int. Ed. Engl. 32:543; Zuckermann et al. (1992) Chemtracts-Macromol. Chem. 4:80; and Simon et al. (1992) Proc. Nat'l. Acad. Sci. USA 89:9367). In a peptoid backbone, the sidechain is attached to the backbone nitrogen atoms rather than the carbon atoms. An example of a suitable peptoid backbone is poly-(N-substituted) glycine (poly-NSG). Synthesis of peptoids is described in, for example, U.S. Pat. No. 5,877,278. As the term is used herein, transporters that have a peptoid backbone are considered “non-peptide” transporters because the transporters are not composed of amino acids having naturally occurring sidechain locations.

Studies carried out in support of the present invention have utilized polypeptides (e.g., peptide backbones). However, other backbones, such as those described above, can provide enhanced biological stability (for example, resistance to enzymatic degradation in vivo).

### Synthesis of Delivery-Enhancing Transport Molecules

Delivery-enhancing transporters can be constructed by any method known in the art. Exemplary peptide polymers can be produced synthetically, preferably using a peptide synthesizer (e.g., an Applied Biosystems Model 433).

N-methyl and hydroxy-amino acids can be substituted for conventional amino acids in solid phase peptide synthesis. However, production of delivery-enhancing transporters with reduced peptide bonds requires synthesis of the dimer of amino acids containing the reduced peptide bond. Such dimers are incorporated into polymers using standard solid phase synthesis procedures. Other synthesis procedures are well known and can be found, for example, in Fletcher et al. (1998) Chem. Rev. 98:763, Simon et al. (1992) Proc. Nat'l. Acad. Sci. USA 89:9367, and references cited therein.

As noted above, the delivery-enhancing transporters of the invention can be flanked by, or interrupted by, one or more non-guanidino/non-amidino subunits (such as glycine, alanine, and cysteine, for example), or a linker (such as an aminocaproic acid group), that do not significantly affect the rate of transmembrane transport or trans-tissue layer transport of the corresponding delivery-enhancing transport/compound composition. Also, any free amino terminal group can be capped with a blocking group, such as an acetyl or benzyl group, to prevent ubiquitination in vivo.

Biologically Active Agents

In the present invention, essentially any biologically active agent or diagnostic molecule can be combined with a delivery-enhancing transporter. In some embodiments, the biologically active agent can be used in its unmodified form, while in other embodiments, the agent will be modified to incorporate a charged (typically acidic) residue to complex with the transporter. The term “biologically active agent” as used herein includes agents in their unmodified form as well as agents that have been modified (e.g., prodrugs) and have reduced levels of activity compared with the parent agent.

The delivery-enhancing transporters can be combined with a wide variety of biologically active agents and molecules that have diagnostic use.

Small Organic Molecules

Small organic molecule therapeutic agents can be advantageously combined with the transporters as described herein, to facilitate or enhance transport of the small molecule compound across one or more layers of an epithelial or endothelial tissue. For example, highly charged agents, such as levodopa (L-3,4-dihydroxy-phenylalanine; L-DOPA) can be combined directly with a delivery-enhancing transporters as described herein and delivered to the desired site. Peptoid and peptidomimetic agents are also contemplated (e.g., Langston (1997) DDT2:255; Giannis et al. (1997) Advances Drug Res. 29:1). Also, the invention is advantageous for delivering small organic molecules that have poor solubilities in aqueous liquids, such as serum and aqueous saline. Thus, compounds whose therapeutic efficacies are limited by their low solubilities can be administered in greater dosages according to the present invention, and can be more efficacious on a molar basis in combined form, relative to the non-combined form, due to higher uptake levels by cells.

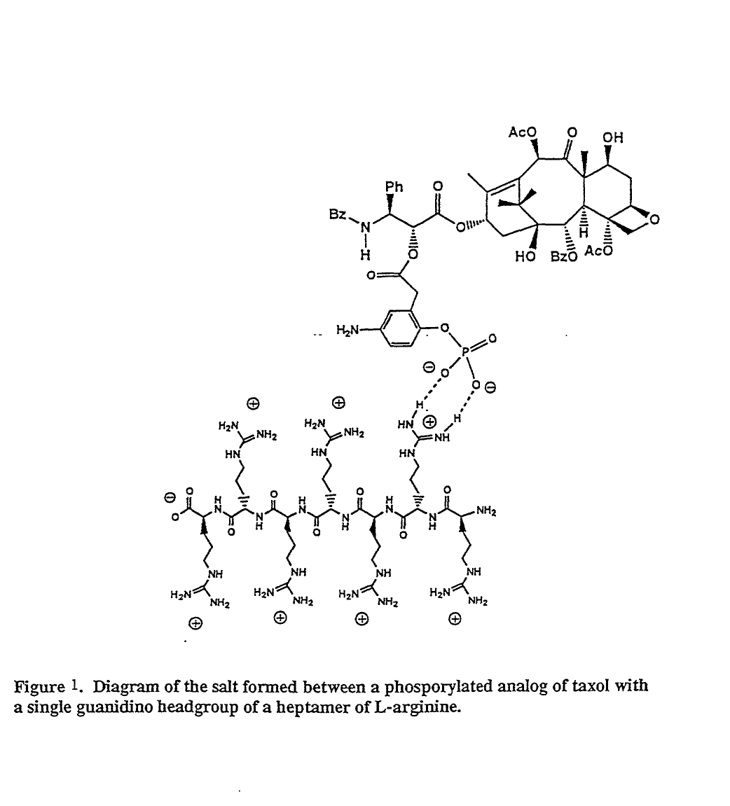

Exemplary of such small organic molecules that form compositions according to the present methods are the taxanes. FIG. 1 illustrates a modified taxane which is combined with a heptamer of arginine to form a delivery-enhancing complex. The complex has enhanced trans-epithelial tissue transport rates relative to corresponding non-complexed forms and is particularly useful for inhibiting growth of cancer cells. Taxanes and taxoids are believed to manifest their anticancer effects by promoting polymerization of microtubules (and inhibiting depolymerization) to an extent that is deleterious to cell function, inhibiting cell replication and ultimately leading to cell death.

As used herein, the term “taxane” refers to paclitaxel (F, R′=acetyl, R″=benzyl, also known under the trademark “TAXOL”) and naturally occurring, synthetic, or bioengineered analogs having a backbone core that contains the A, B, C and D rings of paclitaxel, as illustrated in G. F also indicates the structure of “TAXOTERE™” (R′=H, R″=BOC), which is a somewhat more soluble synthetic analog of paclitaxel sold by Rhone-Poulenc. “Taxoid” refers to naturally occurring, synthetic or bioengineered analogs of paclitaxel that contain the basic A, B and C rings of paclitaxel, as shown in H. Substantial synthetic and biological information is available on syntheses and activities of a variety of taxane and taxoid compounds, as reviewed in Suffness (1995) Taxol: Science and Applications, CRC Press, New York, N.Y., pp. 237-239, particularly in Chapters 12 to 14, as well as in the subsequent paclitaxel literature. Furthermore, a host of cell lines are available for predicting anticancer activities of these compounds against certain cancer types, as described, for example, in Suffness at Chapters 8 and 13.

The delivery-enhancing transporter can be combined with a modified taxane or taxoid which has been modified to include an acid moiety (typically a phosphate). The acid moiety or other charged functional group is conjugated to the taxane or taxoid portion via any suitable site of attachment in the taxane or taxoid. Conveniently, the charged functional group is linked via a C2′-oxygen atom or a C7-oxygen atom, using linking strategies as above. Conjugation of a charged functional group via a C7-oxygen leads to taxane conjugates that have anticancer and antitumor activity despite conjugation at that position. Accordingly, the linker can be cleavable or non-cleavable. Conjugation via the C2′-oxygen significantly reduces anticancer activity, so that a cleavable linker is preferred for conjugation to this site. Other sites of attachment can also be used, such as C10.

It will be appreciated that the taxane and taxoid compositions of the invention have improved water solubility relative to taxol (≈0.25 μg/mL) and taxotere (6-7 μg/mL). Therefore, large amounts of solubilizing agents such as “CREMOPHOR EL” (polyoxyethylated castor oil), polysorbate 80 (polyoxyethylene sorbitan monooleate, also known as “TWEEN 80”), and ethanol are not required. Accordingly, side-effects typically associated with these solubilizing agents, such as anaphylaxis, dyspnea, hypotension, and flushing, can be reduced.

Metals

Metals can be transported into and across one or more layers of epithelial and endothelial tissues using chelating agents such as texaphyrin or diethylene triamine pentacetic acid (DTPA), and a delivery-enhancing transporter. These combinations are useful for delivering metal ions for imaging or therapy. Exemplary metal ions include Eu, Lu, Pr, Gd, ^{99m}Tc, ⁶⁷Ga, ¹¹¹In, ⁹⁰Y, ⁶⁷Cu, and ⁵⁷Co. Preliminary membrane-transport studies with conjugate candidates can be performed using cell-based assays. For example, using europium ions, cellular uptake can be monitored by time-resolved fluorescence measurements. For metal ions that are cytotoxic, uptake can be monitored by cytotoxicity.

Macromolecules

The compositions and methods of the present invention are particularly suited for enhancing transport into and across one or more layers of an epithelial or endothelial tissue for a number of macromolecules, including, but not limited to polypeptides, proteins, polysaccharides, and analogs thereof.

One class of macromolecules that can be transported across one or more layers of an epithelial or endothelial tissue is exemplified by proteins, and in particular, enzymes. Therapeutic proteins include, but are not limited to replacement enzymes. Therapeutic enzymes include, but are not limited to, alglucerase, for use in treating lysozomal glucocerebrosidase deficiency (Gaucher's disease), alpha-L-iduronidase, for use in treating mucopolysaccharidosis I, alpha-N-acetylglucosamidase, for use in treating sanfilippo B syndrome, lipase, for use in treating pancreatic insufficiency, adenosine deaminase, for use in treating severe combined immunodeficiency syndrome, and triose phosphate isomerase, for use in treating neuromuscular dysfunction associated with triose phosphate isomerase deficiency.

In addition, and according to an important aspect of the invention, protein antigens may be delivered to the cytosolic compartment of antigen-presenting cells (APCs), where they are degraded into peptides. The peptides are then transported into the endoplasmic reticulum, where they associate with nascent HLA class I molecules and are displayed on the cell surface. Such “activated” APCs can serve as inducers of class I restricted antigen-specific cytotoxic T-lymphocytes (CTLs), which then proceed to recognize and destroy cells displaying the particular antigen. APCs that are able to carry out this process include, but are not limited to, certain macrophages, B cells and dendritic cells. In one embodiment, the protein antigen is a tumor antigen for eliciting or promoting an immune response against tumor cells. The transport of isolated or soluble proteins into the cytosol of APC with subsequent activation of CTL is exceptional, since, with few exceptions, injection of isolated or soluble proteins does not result either in activation of APC or induction of CTLs. Thus, antigens that are complexed with the transport enhancing compositions of the present invention can serve to stimulate a cellular immune response in vitro or in vivo.

In another embodiment, the invention is useful for delivering immunospecific antibodies or antibody fragments to the cytosol to interfere with deleterious biological processes such as microbial infection. Recent experiments have shown that intracellular antibodies can be effective antiviral agents in plant and mammalian cells (e.g., Tavladoraki et al. (1993) Nature 366:469; and Shaheen et al. (1996) J. Virol. 70:3392. These methods have typically used single-chain variable region fragments (scFv), in which the antibody heavy and light chains are synthesized as a single polypeptide. The variable heavy and light chains are usually separated by a flexible linker peptide (e.g., of 15 amino acids) to yield a 28 kDa molecule that retains the high affinity ligand binding site. The principal obstacle to wide application of this technology has been efficiency of uptake into infected cells. But by complexing transport polymers to scFv fragments, the degree of cellular uptake can be increased, allowing the immunospecific fragments to bind and disable important microbial components, such as HIV Rev, HIV reverse transcriptase, and integrase proteins.

Peptides

Peptides to be delivered by the enhanced transport methods described herein include, but should not be limited to, effector polypeptides, receptor fragments, and the like. Examples include peptides having phosphorylation sites used by proteins mediating intracellular signals. Examples of such proteins include, but are not limited to, protein kinase C, RAF-1, p21Ras, NF-κB, C-JUN, and cytoplasmic tails of membrane receptors such as IL-4 receptor, CD28, CTLA-4, V7, and MHC Class I and Class II antigens.

Diagnostic Imaging and Contrast Agents

The compositions of the present invention are also useful for delivery of diagnostic imaging and contrast agents into and across one or more layers of an epithelial and/or endothelial tissue. Examples of diagnostic agents include substances that are labeled with radioactivity, such as ⁹⁹mTc glucoheptonate, or substances used in magnetic resonance imaging (MRI) procedures such as gadolinium doped chelation agents (e.g. Gd-DTPA). Other examples of diagnostic agents include marker genes that encode proteins that are readily detectable when expressed in a cell (including, but not limited to, (β-galactosidase, green fluorescent protein, luciferase, and the like. A wide variety of labels may be employed, such as radionuclides, fluors, enzymes, enzyme substrates, enzyme cofactors, enzyme inhibitors, ligands (particularly haptens), and the like.

Boron Reagents

The compositions and methods of the present invention are also useful for delivery of boron reagents such as those used in Boron Neutron Capture therapy. In this embodiment, the boron species can be incorporated into the delivery enhancing transporter itself or can be combined with the transporter to more efficiently transfer the boron into a target cell or tissue. Reviews on Boron Neutron Capture can be found as follows: Barth, et al., Mol. Chem. Neuropathol. 21:139-154 (1994); Barth, et al., Cancer Inv. 14:534-550 (1996); Coderre, et al., Radiat. Res. 151:1-18 (1999); Gahbauer, et al., Recent Results CancerRes. 150:183-209 (1998); Hawthorne, Angew. Chem., Int. Ed. Engl. 32:950-984 (1993); Hawthorne, Mol. Med. Today 4:174-181 (1998); Soloway, et al., J. Neuro-Oncol. 33:9-18 (1997); Soloway, et al., Chem. Rev. 98:1515-1562 (1998). For a review on methods to prepare and incorporate boron in amino acids and peptides, see Spielvogel, et al., Phosphorus, Sulfur, Silicon Relat. Elem. 87:267-276 (1994). See also, Cai, et al., J. Med. Chem. 40:3887-3896 (1997).

Therapeutic Agents

In addition to the above classes of biologically active agents, the present invention provides compositions and methods for various classes of therapeutic agents. Illustrative of the agents that can be combined with delivery-enhancing transporters to greatly improve the agent's tissue penetration and efficacy are compounds such as antibacterial agents, antifungal agents, antiviral agents, antiproliferative agents, immunosuppressive agents, vitamins, analgesics, hormones and the like.

Antibacterial agents useful in the present compositions and methods include in general the β-lactam antibiotics and the quinolone antibiotics. More particularly, the agents can be nafcillin, oxacillin, penicillin, amoxacillin, ampicillin, cefotaxime, ceftriaxone, rifampin, minocycline, ciprofloxacin, norfloxacin, erythromycin, vancomycin, or an analog thereof.

Antimicrobial agents useful in the present compositions and methods include in general sulfanilamide, sulfamethoxazole, sulfacetamide, sulfisoxazole, sulfadiazine, penicillins (e.g., penecillins G and V, methicillin, oxacillin, naficillin, ampicillin amoxacillin, carbenicillin, ticarcillin, mezlocillin and piperacillin), cephalosporins (e.g., cephalothin, cefaxolin, cephalexin, cefadroxil, cefamandole, cefoxitin, cefaclor, cefuroxine, loracarbef, cefonicid, cefotetan, ceforanide, cefotaxime, cefpodoxime proxetil, ceftizoxime, cefoperazone, ceftazidime and cefepime), aminoglycosides (e.g., gentamycin, tobramycin, amikacin, netilmicin, neomycin, kanamycin, streptomycin, and the like), tetracyclines (e.g., chlortetracycline, oxytetracycline, demeclocycline, methacycline, doxycycline and minocycline), and macrolides (e.g., erythromycin, clarithromycin, azithromycin).

Antifungal agents useful in the present compositions and methods include in general amphotericin, itraconazole, ketoconazole, miconazole, nystatin, clotrimazole, fluconazole, ciclopirox, econazole, naftifine, terbinafine and griseofulvin.

Antiviral agents useful in the present compositions and methods include in general acyclovir, famciclovir, ganciclovir, foscarnet, idoxuridine, sorivudine, trifluridine, valacyclovir, cidofovir, didanosine, stavudine, zalcitabine, zidovudine, ribavirin and rimantatine.

Antiproliferative and immunosuppressive agents which are useful in the present compositions and methods include methotrexate, azathioprine, fluorouracil, hydroxyurea, 6-thioguanine, chclophosphamide, mechloroethamine hydrochloride, carmustine, cyclosporine, taxol, tacrolimus, vinblastine, dapsone and sulfasalazine.

Histamine receptor agonists and antagonists are another class of agents useful in the present invention. Examples of suitable agents include, 2-methylhistamine, 2-pyridylethylamine, 2-thiazolylethylamine, (R)-α-methylhistamine, impromidine, dimaprit, 4(5)methylhistamine, diphenhydramine, pyrilamine, promethazine, chlorpheniramine, chlorcyclizine, terfenadine, and the like.

Another class of agents useful in the present invention are compounds used in treating asthma. Examples of such agents include the corticosteroids (e.g., beclomethasone, budesonide and prednisone), cromolyn, nedocromil, albuterol, bitolterol mesylate, pirbuterol, salmeterol, terbutyline and theophylline.

Yet another class of biologically active agents which are useful in the present compositions and methods are the vitamins (see GOODMAN & GILMAN'S THE PHARMACOLOGICAL BASIS OF THERAPEUTICS, Ninth Ed. Hardman, et al., eds. McGraw-Hill, p. 1547-1590 (1996)).

A variety of analgesic agents are useful in the present invention including, for example, lidocaine, bupivacaine, novocaine, procaine, tetracaine, benzocaine, cocaine, mepivacaine, etidocaine, proparacaine ropivacaine, prilocaine and the like.

Antineoplastic agents useful in the present compositions and methods include in general pentostatin, 6-mercaptopurine, 6-thioguanine, methotrexate, bleomycins, etoposide, teniposide, dactinomycin, daunorubicin, doxorubicin, mitoxantrone, hydroxyurea, 5-fluorouracil, cytarabine, fludarabine, mitomycin, cisplatin, procarbazine, dacarbazine, paclitaxel, colchicine, the vinca alkaloids, and the like.

Modification of Biologically Active Agents

In some embodiments, the biological agent will be modified to incorporate a functional group (e.g., a carboxylic acid group, a phosphate or phosphate ester, a sulfonic acid group, and the like). Typically, the biological agent will be modified to incorporate a suitable group by attaching the group via a linker to the biological agent. Preferably, the linker will be a cleavable linker which can liberate the biological agent.

1. Chemical Linkages and Self-Cleavable Linkers

Biologically active agents such as small organic molecules and macromolecules can be modified using a number of methods known in the art (see, for example, Wong, S. S., Ed., Chemistry of Protein Conjugation and Cross-Linking, CRC Press, Inc., Boca Raton, Fla. (1991), either directly (e.g., with a carbodiimide) or via a linking moiety. In particular, carbamate, ester, thioether, disulfide, and hydrazone linkages are generally easy to form and suitable for most applications. Ester and disulfide linkages are preferred if the linkage is to be readily degraded in the cytosol, after transport of the substance across the cell membrane.

Various functional groups (hydroxyl, amino, halogen, etc.) present on the biologically active agent can be used as a handle to attach a suitable complexing group. For example, a hydroxyl group can be modified as shown in Scheme 1 to include an acidic phosphate group.

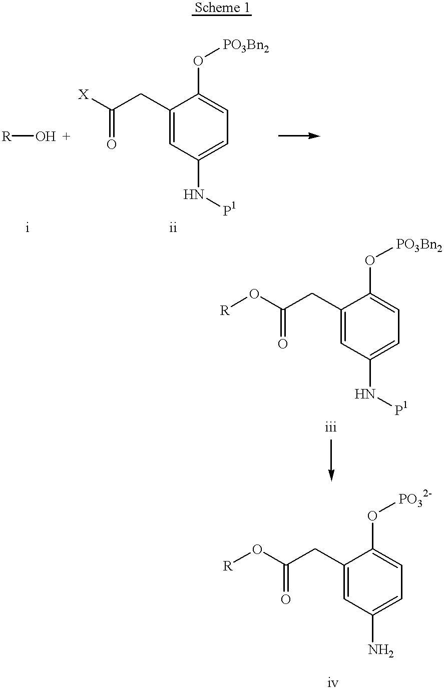

As shown in Scheme 1, essentially any biological agent having a hydroxyl group (i) can be modified with a suitable phosphate-ester-containing phenacyl group (ii, wherein X is a OH or a leaving group such as Cl, and P¹ is a protecting group) to form the derivative iii. Removal of the protecting group P¹ and the phosphate benzyl esters (or other suitable protecting groups) provides a phosphate-containing biological agent derivative (iv).

Derivatized biological agents can then be combined with a suitable delivery-enhancing transporter to form a non-covalently bound complex which is suitable to delivery of the biological agent in vivo. After administration of the complex, an endogenous phosphatase enzyme cleaves the phosphate moiety from the derivatized biological agent and the phenolic hydroxy group which is liberated, then cyclized onto the ester carbonyl (shown in iv), to liberate the biological agent in an underivatized form.

One of skill in the art will appreciate that this approach will find broad applicability to essentially any biological agent having a hydroxyl group (other than those which are so sterically encumbered as to be unavailable for reaction).

A variety of other linking groups and acidic moieties are useful for derivatizing biological agents. A discussion of these groups and their use can be found in, for example, Senter, et al., J. Org. Chem. 55:2975-78 (1990) and Koneko, et al., Bioconjugate Chem. 2:133-141 (1991). One of skill in the art will appreciate that certain groups are preferred for modifying amino groups available on a biological agent, while other groups will be preferred for modifying thiol groups on biological agents. Still other groups will be more preferred for modifying hydroxy groups on biological agents. In some embodiments, a biological agent may not have a functional group for modification, but can be first modified to incorporate a hydroxy, amino, or thiol substituent. Preferably, the substituent is provided in a non-interfering portion of the biological agent.

2. Other Cleavable Linkers

In certain embodiments, the biologically active agents are modified by attaching a charged functional group to the agent using a linkage that is specifically cleavable or releasable. The use of such linkages is particularly important for biologically active agents that are inactive in any form other than their unmodified form. As used herein, “specifically cleavable” refers to the linkage between the charged functional group and the agent being cleaved. The linkage is preferably a readily cleavable linkage, meaning that it is susceptible to enzymatic or solvent-mediated cleavage in vivo. For this purpose, linkers containing carboxylic acid esters and disulfide bonds are sometimes preferred, where the former groups are hydrolyzed enzymatically or chemically, and the latter are severed by disulfide exchange, e.g., in the presence of glutathione. The linkage can be selected so that it is cleavable by an enzymatic activity that is known to be present in one or more of the layers of an epithelial or endothelial tissue. For example, the stratum granulosum of skin has a relatively high concentration of N-peptidase activity.

In some embodiments, a specifically cleavable linker can be engineered onto the biological agent. For example, amino acids that constitute a protease recognition site or other such specifically recognized enzymatic cleavage site can be used to link the charged functional group to the agent. Alternatively, chemical or other types of linkers that are cleavable by, for example, exposure to light or other stimulus can be used to link the charged functional group to the agent of interest.

### Compositions of Delivery-Enhancing Transporters and Biologically Active Agents

The agent to be transported can be combined with the delivery-enhancing transporter according to a number of embodiments. In one preferred embodiment, the agent is combined with a single delivery-enhancing transporter to form a composition which is thought to exist as a non-covalently bound ion pair.

In a second embodiment, the agent is combined with more than one delivery-enhancing transporter, in the same manner as above.

In a third embodiment, the composition contains two agent moieties in combination with a single delivery-enhancing transporter. For this embodiment, it is presently preferred that the agent has a molecular weight of less than 10 kDa.

In a fourth embodiment, the agent is modified or derivatized to include a charged group which can participate in forming an ion pair with the delivery-enhancing transporter.

Since a significant portion of the topological surface of a small molecule is often involved, and therefore required, for biological activity, the small molecule agents will preferably be in unmodified form, or modified to include a charged functional group attached to the molecule via a cleavable linker.

Typically, the compositions of the invention can be prepared by combining the components (delivery-enhancing transporter and biologically active agents) in a suitable medium and concentrating the composition to dryness. In many embodiments, the compositions are formed in water or a buffered aqueous solution, lyophilized and packaged for reconstitution and use by the clinician. Alternatively, compositions can be prepared immediately prior to use. In still other embodiments, the compositions will be prepared by combining the components in the medium to be used for administration.

According to an important aspect of the present invention, it has been found by the applicants that association of a single delivery-enhancing transporter to any of a variety of types of biologically active agents is sufficient to substantially enhance the rate of uptake of an agent into or across a biological barrier such as one or more layers of epithelial and endothelial tissues. Additionally, the transporters described herein do not require the presence of a large hydrophobic moiety in the associated complex. In fact, the use of a large hydrophobic moiety can significantly impede or prevent cross-layer transport in epithelial or endothelial tissue due to adhesion of the hydrophobic moiety to the lipid bilayer of cells that make up the epithelial or endothelial tissue. Accordingly, the compositions of the present invention are in one embodiment, substantially free of hydrophobic moieties, such as lipid and fatty acid molecules.

### Uses of Delivery-Enhancing Transporter and Biological Agent Compositions

The delivery-enhancing transporters in combination with certain biologically active agents find use in therapeutic, prophylactic and diagnostic applications. The delivery-enhancing transporters can carry a diagnostic or biologically active reagent to the surface of, into or across a biological barrier, including one or more layers of skin or other epithelial tissue (e.g., gastrointestinal, lung, and the like), or across endothelial tissues such as the blood brain barrier. This property makes the compositions useful for treating conditions by delivering agents that must penetrate across one or more tissue layers in order to exert their biological effect.

Compositions and methods of the present invention have particular utility in the area of human and veterinary therapeutics. Generally, administered dosages will be effective to deliver picomolar to micromolar concentrations of the therapeutic composition to the effector site. Appropriate dosages and concentrations will depend on factors such as the therapeutic composition or drug, the site of intended delivery, and the route of administration, all of which can be derived empirically according to methods well known in the art. Further guidance can be obtained from studies using experimental animal models for evaluating dosage, as are known in the art.

Administration of the compounds of the invention with a suitable pharmaceutical excipient as necessary can be carried out via any of the accepted modes of administration. Thus, administration can be, for example, intravenous, topical, subcutaneous, transcutaneous, intramuscular, oral, intra-joint, parenteral, peritoneal, intranasal, or by inhalation. Suitable sites of administration thus include, but are not limited to, skin, bronchial, gastrointestinal, anal, vaginal, eye, and ear. The formulations may take the form of solid, semi-solid, lyophilized powder, or liquid dosage forms, such as, for example, tablets, pills, capsules, powders, solutions, suspensions, emulsions, suppositories, retention enemas, creams, ointments, lotions, aerosols or the like, preferably in unit dosage forms suitable for simple administration of precise dosages.

The compositions typically include a conventional pharmaceutical carrier or excipient and may additionally include other medicinal agents, carriers, adjuvants, and the like. Preferably, the composition will be about 5% to 75% by weight of a compound or compound/transporter combination of the invention, with the remainder consisting of suitable pharmaceutical excipients. Appropriate excipients can be tailored to the particular composition and route of administration by methods well known in the art, e.g., (REMINGTON'S PHARMACEUTICAL SCIENCES, 18TH ED., Mack Publishing Co., Easton, Pa. (1990).

For oral administration, such excipients include pharmaceutical grades of mannitol, lactose, starch, magnesium stearate, sodium saccharine, talcum, cellulose, glucose, gelatin, sucrose, magnesium carbonate, and the like. The composition may take the form of a solution, suspension, tablet, pill, capsule, powder, sustained-release formulation, and the like.

In some embodiments, the pharmaceutical compositions take the form of a pill, tablet or capsule, and thus, the composition can contain, along with the biologically active conjugate, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof.

The active compounds of the formulas may be formulated into a suppository comprising, for example, about 0.5% to about 50% of a compound of the invention, disposed in a polyethylene glycol (PEG) carrier (e.g., PEG 1000 [96%] and PEG 4000 [4%]).

Liquid compositions can be prepared by dissolving or dispersing compound (about 0.5% to about 20%), and optional pharmaceutical adjuvants in a carrier, such as, for example, aqueous saline (e.g., 0.9% w/v sodium chloride), aqueous dextrose, glycerol, ethanol and the like, to form a solution or suspension, e.g., for intravenous administration. The active compounds may also be formulated into a retention enema.

If desired, the composition to be administered may also contain minor amounts of non-toxic auxiliary substances such as wetting or emulsifying agents, pH buffering agents, such as, for example, sodium acetate, sorbitan monolaurate, or triethanolamine oleate.

For topical administration, the composition is administered in any suitable format, such as a lotion or a transdermal patch. For delivery by inhalation, the composition can be delivered as a dry powder (e.g., Inhale Therapeutics) or in liquid form via a nebulizer.

Methods for preparing such dosage forms are known or will be apparent to those skilled in the art; for example, see Remington's Pharmaceutical Sciences, supra., and similar publications. The composition to be administered will, in any event, contain a quantity of the pro-drug and/or active compound(s) in a pharmaceutically effective amount for relief of the condition being treated when administered in accordance with the teachings of this invention.

Generally, the compounds or biological agents used in the invention are administered in a therapeutically effective amount, i.e., a dosage sufficient to effect treatment, which will vary depending on the individual and condition being treated. Typically, a therapeutically effective daily dose is from 0.1 to 100 mg/kg of body weight per day of drug. Most conditions respond to administration of a total dosage of between about 1 and about 30 mg/kg of body weight per day, or between about 70 mg and 2100 mg per day for a 70 kg person.

Stability of the compound/transporter composition can be further controlled by the nature and stereochemistry of the backbone and sidechains of the delivery-enhancing transporters. For polypeptide delivery-enhancing transporters, D-isomers are generally resistant to endogenous proteases, and therefore have longer half-lives in serum and within cells. D-polypeptide polymers are therefore appropriate when longer duration of action is desired. L-polypeptide polymers have shorter half-lives due to their susceptibility to proteases, and are therefore chosen to impart shorter acting effects. This allows side-effects to be averted more readily by withdrawing therapy as soon as side-effects are observed. Polypeptides comprising mixtures of D and L-residues have intermediate stabilities. Homo-D-polymers are generally preferred.

Application to Skin

The delivery-enhancing transporters of the invention make possible the delivery of biologically active and diagnostic agents across the skin. Surprisingly, the transporters can deliver an agent across the stratum corneum, which previously had been a nearly impenetrable barrier to drug delivery. The stratum corneum, the outermost layer of the skin, is composed of several layers of dead, keratin-filled skin cells that are tightly bound together by a “glue” composed of cholesterol and fatty acids. Once the agents are delivered through the stratum corneum by the transporters of the invention, the agents can enter the viable epidermis, which is composed of the stratum granulosum, stratum lucidum and stratum germinativum which, along with the stratum corneum, make up the epidermis. Delivery in some embodiments of the invention is through the epidermis and into the dermis, including one or both of the papillary dermis and the reticular dermis.

This ability to obtain penetration of one or more layers of the skin can greatly enhance the efficacy of compounds such as antibacterials, antifungals, antivirals, antiproliferatives, immunosuppressives, vitamins, analgesics, hormones, and the like. Numerous such compounds are known to those of skill in the art (see, e.g., Hardman and Limbird, Goodman & Gilman's The Pharmacological Basis of Therapeutics, McGraw-Hill, New York, 1996).

In some embodiments, the agent is delivered into a blood vessel that is present in the epithelial tissue, thus providing a means for delivery of the agent systemically. Delivery can be either intrafollicular or interfollicular, or both. Pretreatment of the skin is not required for delivery of the conjugates.

In other embodiments, the delivery-enhancing transporters are useful for delivering cosmetics and agents that can treat skin conditions. Target cells in the skin that are of interest include, for example, fibroblasts, epithelial cells and immune cells. For example, the transporters provide the ability to deliver compounds such as antiinflammatory agents to immune cells found in the dermis.

Glucocorticoids are among the compounds for which delivery across skin can be enhanced by the delivery-enhancing transporters of the invention. Compositions of glucocorticoids and delivery-enhancing transporters are useful for treating inflammatory skin diseases, for example. Examples of particular conditions include bullous disease, collagen vascular diseases, sarcoidosis, Sweet's disease, pyoderma gangrenosum, Type I reactive leprosy, capillary hemangiomas, contact dermatitis, atopic dermatitis, lichen planus, exfoliative dermatitis, erythema nodosum, hormonal abnormalities (including acne and hirsutism), as well as toxic epidermal necrolysis, erythema multiforme, cutaneous T-cell lymphoma, discoid lupus erythematosus, and the like.

Retinoids are another example of a biologically active agent for which one can use the delivery-enhancing transporters of the invention to enhance delivery into and across one or more layers of the skin or other epithelial or endothelial tissue. Retinoids that are presently in use include, for example retinol, tretinoin, isotretinoin, etretinate, acitretin, and arotinoid. Conditions that are treatable using retinoids in combination with the delivery-enhancing transporters described herein include, but are not limited to, acne, keratinization disorders, skin cancer, precancerous conditions, psoriasis, cutaneous aging, discoid lupus erythematosus, scleromyxedema, verrucous epidermal nevus, subcorneal pustular dermatosis, Reiter's syndrome, warts, lichen planus, acanthosis nigricans, sarcoidosis, Grover's disease, porokeratosis, and the like.

Cytotoxic and immunosuppressive drugs constitute an additional class of drugs for which the delivery-enhancing transporters of the invention are useful. These agents are commonly used to treat hyperproliferative diseases such as psoriasis, as well as for immune diseases such as bullous dermatoses and leukocytoclastic vasculitis. Examples of such compounds that one can combine with the delivery-enhancing transporters of the invention include, but are not limited to, antimetabolites such as methotrexate, azathioprine, fluorouracil, hydroxyurea and 6-thioguanine. Other examples are alkylating agents such as cyclophosphamide, mechloroethamine hydrochloride, carmustine. Cyclosporine, taxol, tacrolimus and vinblastine are additional examples of useful biological agents, as are dapsone and sulfasalazine.

The delivery-enhancing transporters can be combined with agents that are useful for treating conditions such as lupus erythematosus (both discoid and systemic), cutaneous dermatomyositis, porphyria cutanea tarda and polymorphous light eruption. Agents useful for treating such conditions include, for example, quinine, chloroquine, hydroxychloroquine, and quinacrine.

The delivery-enhancing transporters of the invention are also useful for transdermal delivery of antiinfective agents. For example, antibacterial, antifungal and antiviral agents can be combined with the delivery-enhancing transporters. Antibacterial agents are useful for treating conditions such as acne, cutaneous infections, and the like. Antifungal agents can be used to treat tinea corporis, tinea pedis, onychomycosis, candidiasis, tinea versicolor, and the like. Because of the delivery-enhancing properties of the combinations, these compositions are useful for treating both localized and widespread infections. Antifungal agents are also useful for treating onychomycosis. Examples of antiviral agents include, but are not limited to, acyclovir, famciclovir, gancyclovir and valacyclovir.

Another example of a biologically active agent for which enhancement of delivery by combination with the delivery-enhancing transporters of the invention is desirable are the antihistamines. These agents are useful for treating conditions such as pruritus due to urticaria, atopic dermatitis, contact dermatitis, psoriasis, and many others. Examples of such reagents include, for example, terfenadine, astemizole, lorotadine, cetirizine, acrivastine, temelastine, cimetidine, ranitidine, famotidine, nizatidine, and the like. Tricyclic antidepressants can also be delivered using the delivery-enhancing transporters of the invention.

Topical antipsoriasis drugs are also of interest. Agents such as corticosteroids, calcipotriene, and anthralin can also be combined with the delivery-enhancing transporters of the invention and applied to skin.

The delivery-enhancing transporters of the invention are also useful for enhancing delivery of photochemotherapeutic agents into and across one or more layers of skin and other epithelial tissues. Such compounds include, for example, the psoralens, and the like. Sunscreen components are also of interest; these include p-aminobenzoic acid esters, cinnamates and salicylates, as well as benzophenones, anthranilates, and avobenzone.

Pain relief agents and local anesthetics constitute another class of compounds for which combination with a delivery-enhancing transporter can enhance treatment. Lidocaine, bupibacaine, novocaine, procaine, tetracaine, benzocaine, cocaine, and the opiates, are among the compounds that one can combine with the delivery-enhancing transporters of the invention.

Other biological agents of interest include, for example, minoxidil, keratolytic agents, destructive agents such as podophyllin, hydroquinone, capsaicin, masoprocol, colchicine, and gold.

Gastrointestinal Administration

The compositions of the present invention are also useful for delivery of drugs by gastrointestinal administration. Gastrointestinal administration can be used for both systemically active drugs, and for drugs that act in the gastrointestinal epithelium.

Among the gastrointestinal conditions that are treatable using appropriate reagents combined with the delivery-enhancing transporters are Crohn's disease (e.g., cyclosporin and FK506), ulcerative colitis, gastrointestinal ulcers, peptic ulcer disease, imbalance of salt and water absorption (can lead to constipation, diarrhea, or malnutrition), abnormal proliferative diseases, and the like. Ulcer treatments include, for example, drugs that reduce gastric acid secretion, such as H₂ histamine inhibitors (e.g., cymetidine and ranitidine) and inhibitors of the proton-potassium ATPase (e.g., lansoprazle amd omeprazle), and antibiotics directed at Helicobacter pylori.

Antibiotics are among the biologically active agents that are useful when combined with a delivery-enhancing transporter, particularly those that act on invasive bacteria, such as Shigella, Salmonella, and Yersinia. Such compounds include, for example, norfloxacin, ciprofloxacin, trimethoprim, sulfamethyloxazole, and the like.

Anti-neoplastic agents can also be combined with a delivery-enhancing transporter as described herein and administered by the gastrointestinal route. Suitable agents include, for example, cisplatin, methotrexate, taxol, fluorouracil, mercaptopurine, donorubicin, bleomycin, and the like.

Respiratory Tract Administration

The compositions of the invention can also used to enhance administration of drugs through the respiratory tract. The respiratory tract, which includes the nasal mucosa, hypopharynx, and large and small airway structures, provides a large mucosal surface for drug absorption. The enhanced penetration of the complexed agents into and across one or more layers of the epithelial tissue that is provided by the delivery-enhancing transporters results in amplification of the advantages that respiratory tract delivery has over other delivery methods. For example, lower doses of an agent are often needed to obtain a desired effect, a local therapeutic effect can occur more rapidly, and systemic therapeutic blood levels of the agent are obtained quickly. Rapid onset of pharmacological activity can result from respiratory tract administration. Moreover, respiratory tract administration generally has relatively few side effects.

The compositions the present invention can be used to deliver biological agents that are useful for treatment of pulmonary conditions. Examples of conditions treatable by nasal administration include, for example, asthma. Suitable biological agents include antiinflammatory agents, such as corticosteroids, cromolyn, and nedocromil, bronchodialators such as β2-selective adronergic drugs and theophylline, and immunosuppressive drugs (e.g., cyclosporin and FK506). Other conditions include, for example, allergic rhinitis (which can be treated with glucocorticoids), and chronic obstructive pulmonary disease (emphysema). Other drugs that act on the pulmonary tissues and can be delivered using the transporters of the invention include beta-agonists, mast cell stabilizers, antibiotics, antifungal and antiviral agents, surfactants, vasoactive drugs, sedatives and hormones.

Respiratory tract administration is useful not only for treatment of pulmonary conditions, but also for delivery of drugs to distant target organs via the circulatory system. A wide variety of such drugs and diagnostic agents can be administered through the respiratory tract after combination with the delivery-enhancing transporters as described herein.

Delivery of Agents across the Blood Brain Barrier

The compositions of the present invention are also useful for delivering biologically active and diagnostic agents across the blood brain barrier. The agents are useful for treating ischemia (e.g., using an anti-apoptotic drug), as well as for delivering neurotransmitters and other agents for treating various conditions such as schizophrenia, Parkinson's disease, and pain (e.g., morphine, the opiates). The 5-hydroxytryptamine receptor antagonist is useful for treating conditions such as migraine headaches and anxiety.

### EXAMPLES

The following examples are offered to illustrate, but not to limit the present invention.

### Example 1

This example illustrates the ability of polyArg to facilitate cellular uptake of small organic acids.

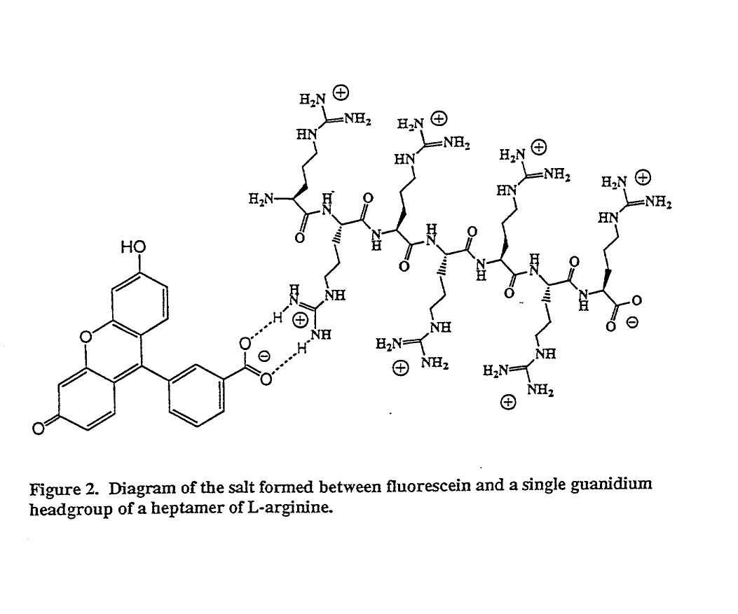

The ability to form complexes between the polymers containing multiple guanidinium groups and small organic acids was examined, along with the ability of the polymer to assist in cellular uptake of the organic acid. In separate vials, n equivalents (with n=1 to 6) of fluorescein, an acidic compound normally poorly soluble in water, were added to the free base of a nonamer of arginine in water (schematically shown in FIG. 2). To neutralize the compound, 6-n equivalents of phosphoric acid were subsequently added to each flask and the solutions were frozen and lyophilized. When the dried powders were taken up in phosphate buffered saline they were very water soluble and intensely yellow. Elemental analysis confirmed that the eight compounds differed in their fluorescein:peptide ratio from 1:1 to 6:1.

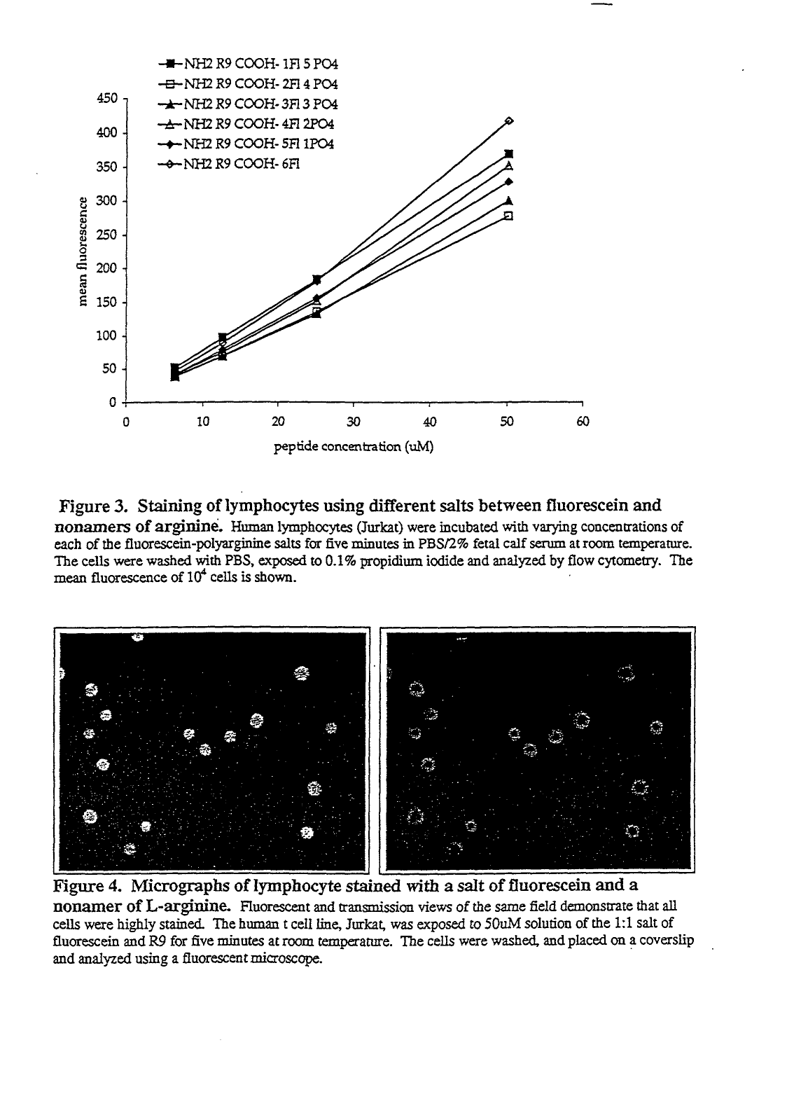

When dilutions of each of the solutions (normalized for fluorescein concentration by measuring their absorption at 490 nm and using the extinction coefficient of fluorescein to calculate the molarity) were used in cellular uptake assays, the resultant cells were stained equivalently within experimental error (see FIG. 3). This result indicates that all the molecules of fluorescein were deposited on the cell surface, regardless of whether they were part of a 1:1 or a 1:6 peptide:fluorescein salt. However, the staining pattern of the cells was fundamentally different when compared to fluorescein that was covalently attached to short polymers of arginine (see FIG. 4). Distinct punctate staining was seen on the cell surface as well as in the cytosol, when covalent conjugates were used (data not shown). More importantly, staining of individual cells was very heterogeneous, with the variation in cell fluorescence ranging over three orders of magnitude. In contrast, when noncovalent conjugates were used, cell staining was remarkably uniform with cell fluorescence varying only by a factor of 2-4. The staining was extremely intense, with the majority of the dye being on the cell surface (see FIG. 4).

### Example 2

This example provides a synthesis for a phosphate-cleavable taxol conjugate which is useful in complexes described herein.

[chemical structure]

2.1 To a suspension of o-hydroxy phenylacetic acid (15.0 g, 0.099 mol) in H₂ O (39 mL) at 0° C. was added a solution of nitric acid (12 mL of 65% in 8 mL H₂O) slowly via pipette. The solution was stirred for an additional 1.5 h at 0° C. The mixture was then warmed to ambient temperature and allowed to stir for an additional 0.5 h. The heterogenous solution was poured over ice (10 g) and filtered to remove the insoluble ortho-nitro isomer. The reddish solution was concentrated under reduced pressure, and the thick residue was redissolved in 6N HCl and filtered through celite. The solvent was again removed under reduced pressure to provide the desired 2-hydroxy-5-nitro-phenylacetic acid as a light, brownish-red solid (40% yield). The product (II-a) was used in the next step without further purification.

2.2 Product II-a (765 mg, 3.88 mmol) was dissolved in freshly distilled THF (5 mL) under argon atmosphere. The solution was cooled to 0° C., and borane-THF (1.0 M in THF, 9.7 mL, 9.7 mmol, 2.5 eq) was added dropwise via syringe with apparent evolution of hydrogen. The reaction was permitted to stir for an additional 16 h, slowly warming to room temperature. The reaction was quenched by slow addition of 1M HCl (with furious bubbling) and 10 mL of ethyl acetate. The layers were separated and the aqueous layer extracted five times with ethyl acetate. The combined organic layers were washed with brine and dried over magnesium sulfate. The solvent was evaporated in vacuo and the residue purified by rapid column chromatography (1:1 hexane:ethyl acetate) to provide the desired nitro-alcohol (II-b) as a light yellow solid (85% yield).

2.3 Nitro-alcohol (II-b) (150 mg, 0.819 mmol) was dissolved in dry DMF (5 mL) containing di-t-butyl dicarbonate (190 mg, 1.05 eq) and 10% Pd/C (10 mg). The mixture was placed in a Parr apparatus and pressurized/purged five times. The solution was then pressurized to 47 psi and allowed to shake for 24 h. The reaction was quenched by filtration through celite, and the solvent was removed under reduced pressure. The residue was purified by column chromatography (1:1 hexane:ethyl acetate) to provide the protected aniline product (II-c) as a tan crystalline solid in 70% yield.

2.4 TBDMS-Cl (48 mg, 0.316 mmol) was dissolved in freshly distilled dichloromethane (4 mL) under an argon atmosphere. To this solution was added imidazole (24 mg, 0.347 mmol, 1.1 eq) and immediately a white precipitate formed. The solution was stirred for 30 min at room temperature, at which point product II-c (80 mg, 0.316 mmol, 1.0 eq) was added rapidly as a solution in dichloromethane/THF (1.0 mL). The resulting mixture was permitted to stir for an additional 18 h at ambient temperature. Reaction was quenched by addition of saturated aqueous ammonium chloride. The layers were separated and the aqueous phase extracted 3 times with ethyl acetate and the combined organic layers washed with brine and dried over sodium sulfate. The organic phase was concentrated to provide silyl ether-phenol product (II-d) as a light yellow oil (90% yield).

2.5 Silyl ether-phenol II-d (150 mg, 0.408 mmol) was dissolved in freshly distilled THF (7 mL) under argon and the solution cooled to 0° C. n-BuLi (2.3 M in hexane, 214 uL) was then added dropwise via syringe. A color change from light yellow to deep red was noticed immediately. After 5 min, tetrabenzyl pyrophosphate (242 mg, 0.45 mmol 1.1 eq) was added rapidly to the stirring solution under argon. The solution was stirred for an additional 18 h under inert atmosphere, slowly warming to room temperature, during which time a white precipitate forms. The reaction was quenched by addition of saturated aqueous ammonium chloride and 10 mL of ethyl acetate. The layers were separated, and the aqueous layer was extracted 5 times with ethyl acetate. The combined organic phases were washed with brine and dried over magnesium sulfate. The solvent was removed by evaporation and the residue purified by rapid column chromatography (1:1 hexane:ethyl acetate) to provide the desired phosphate-silyl ether (II-e) as a light orange oil (90% yield).

2.6 Phosphate-silyl ether (II-e) (10 mg, 0.0159 mmol) was dissolved in 2 mL of dry ethanol at room temperature. To the stirring solution was added 20 uL of conc. HCl (1% v:v solution), and the mixture was permitted to stir until TLC analysis indicated the reaction was complete. Solid potassium carbonate was added to quench the reaction, and the mixture was rapidly filtered through silica gel and concentrated to give crude alcohol-dibenzyl phosphate product (II-f) as a light yellow oil (100% yield).

2.7 Alcohol II-f (78 mg, 0.152 mmol) was dissolved in freshly distilled dichloromethane (10 mL) under an argon atmosphere. To the solution was added Dess-Martin periodinane (90 mg, 0.213 mmol, 1.4 eq). The solution was permitted to stir, and the progress of the reaction was monitored by TLC analysis. Once TLC indicated completion, reaction was quenched by addition of 1:1 saturated aqueous sodium bicarbonate:saturated aqueous sodium thiosulfite. The biphasic mixture was permitted to stir for 1 h at ambient temperature. The layers were separated, and the aqueous phase was extracted 3 times with ethyl acetate. The combined oragnic layers were washed with brine and dried over sodium sulfate. Solvent was removed under reduced pressure to provide aldehyde product (II-g) as a light tan oil (100% yield).

2.8 Aldehyde II-g (78 mg, 0.152 mmol) was dissolved in t-butanol/water (3.5 mL) under inert atmosphere. To the rapidly stirring solution was added 2-methyl-2-butene (1.0 M in THF, 1.5 mL), sodium phosphate-monobasic (105 mg, 0.76 mmol, 5 eq) and sodium chlorite (69 mg, 0.76 mmol, 5 eq). The solution was permitted to stir for 8 additional hours at room temperature. The solution was concentrated, and the residue was acidified and extracted with ethyl acetate 3 times. The combined organic phases were dried over magnesium sulfate. The solution was again concentrated under reduced pressure and the residue was purified via column chromatography (2:1 ethyl acetate:hexane) to give the desired carboxylic acid-dibenzylphosphate (II-h) as a light yellow oil (65% yield).

2.9 Acid II-h (8.0 mg, 0.0152 mmol, 1.1 eq) was dissolved in freshly distilled dichloromethane (2 mL) under argon at ambient temperature. To this mixture was added paclitaxel (12 mg, 0.0138 mmol, 1 eq) followed by DMAP (2 mg, 0.0138 mmol, 1 eq) and DCC (3.2 mg, 0.0152, 1.1 eq). The mixture was allowed to stir at room temperature for an additional 4 h, during which a light precipitate formed. Once TLC analysis indicated that the reaction was complete, solvent was removed under reduced pressure, and the residue was purified by rapid column chromatography (1:1 hexane:ethyl acetate) to provide paclitaxel-C2′-carboxylate ester (II-i) as a white, crystalline solid (65% yield).

2.10 Ester II-i (5.0 mg) was dissolved in neat formic acid (1.0 mL) under an argon atmosphere at room temperature and permitted to stir for 30 min. Once TLC indicated that the reaction was complete, the solution was concentrated under reduced pressure and the residue purified by rapid filtration through silica gel to give the desired aniline-taxol compound (II-j) in 50% yield as a white powder.

[chemical structure]

### Example 3

This example illustrates the delivery to a cell of therapeutically useful amounts of taxol, using polymers containing multiple guanidinium salts.

To determine whether a therapeutically useful amount of a drug can be delivered into cells using noncovalent salts, one equivalent of a modified taxol containing a phosphate group was added to the free base of a heptamer of arginine, neutralized using four equivalents of phosphoric acid, frozen, and lyophilized.

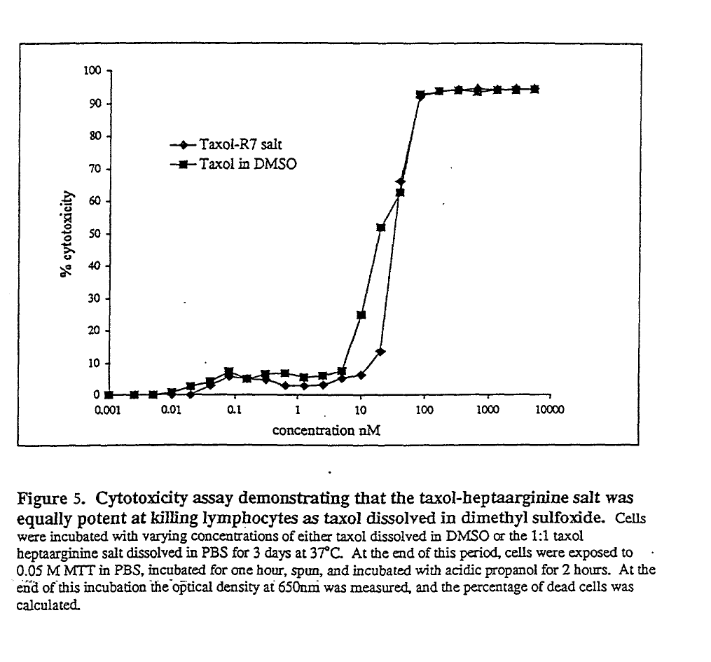

In contrast with taxol itself, which has very limited solubility in water, the salt was freely soluble. This water soluble analog of taxol was assayed for biological activity using a standard cytotoxicity assay. When directly compared with unmodified taxol dissolved in DMSO, the salt was equally potent (see FIG. 5). This remarkable result demonstrates that not only did salt formation between the phosphate analog of taxol and a heptamer of arginine dramatically increase water solubility, but it also could effectively deliver therapeutic amounts of taxol intracellularly. The earlier results with fluorescein indicate that the taxol most likely was delivered to the cell surface, from where it partitioned into the cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for delivery of a compound to the surface of, into or across a biological barrier, the method comprising contacting the barrier with a composition comprising the compound and a delivery-enhancing transporter,
wherein the delivery-enhancing transporter comprises sufficient guanidino or amidino moieties to increase delivery of the compound into or across the barrier compared to delivery of the compound in the absence of the delivery-enhancing transporter.

2. The method of claim 1, wherein the delivery-enhancing transporter comprises a peptide backbone.

3. The method of claim 1, wherein the delivery-enhancing transporter comprises a non-peptide backbone.

4. The method of claim 1, wherein the delivery-enhancing transporter comprises from 6 to 50 guanidino or amidino moieties.

5. The method of claim 4, wherein the delivery-enhancing transporter comprises from 7 to 15 guanidino moieties.

6. The method of claim 1, wherein the delivery-enhancing transporter comprises at least 6 contiguous subunits which each include a guanidino or amidino moiety.

7. The method of claim 1, wherein the delivery-enhancing transporter comprises from 6 to 50 subunits, at least 50% of which include a guanidino or amidino moiety.

8. The method of claim 7, wherein at least about 70% of the subunits in the delivery-enhancing transporter include a guanidino moiety.

9. The method of claim 7, wherein each subunit includes a guanidino moiety.

10. The method of claim 7, wherein the subunits are selected from the group consisting of L-arginine, D-arginine, L-homoarginine and D-homoarginine residues.

11. The method of claim 10, wherein each subunit is independently a D- or L-arginine residue.

12. The method of claim 11, wherein at least one subunit is D-arginine.

13. The method of claim 12, wherein all of the arginine residues have a D-configuration.

14. The method of claim 1, wherein the compound is a modified biological agent.

15. The method of claim 1, wherein the composition comprises at least two delivery-enhancing transporters.

16. The method of claim 1, wherein the barrier is an intact epithelial or endothelial tissue layer or layers.

17. The method of claim 1, wherein the compound is a diagnostic imaging or contrast agent.

18. The method of claim 1, wherein the compound is a non-nucleic acid.

19. The method of claim 1, wherein the compound is a non-polypeptide.

20. The method of claim 1, wherein the compound is selected from the group consisting of antibacterials, antifungals, antivirals, antiproliferatives, immunosuppressives, vitamins, analgesics, and hormones.

21. The method of claim 1, wherein the biological barrier is skin.

22. The method of claim 21, wherein the compound is delivered into and across one or more of the stratum corneum, stratum granulosum, stratum lucidum and stratum germinativum.

23. The method of claim 21, wherein the compound crosses the stratum corneum in the absence of skin pretreatment.

24. The method of claim 21, wherein the composition is administered topically and the compound is taken up by cells that comprise the follicular or interfollicular epidermis.

25. The method of claim 21, wherein the composition is administered by a transdermal patch.

26. The method of claim 1, wherein the compound is a therapeutic agent for a condition selected from the group consisting of Crohn's disease, ulcerative colitis, gastrointestinal ulcers, peptic ulcer disease, and abnormal proliferative diseases.

27. The method of claim 26, wherein the compound is a therapeutic for ulcers and is selected from the group consisting of an H₂ histamine inhibitor, an inhibitor of the proton-potassium ATPase, and an antibiotic directed at Helicobacter pylori.

28. The method of claim 1, wherein the compound is a therapeutic agent for treating a bronchial condition selected from the group consisting of cystic fibrosis, asthma, allergic rhinitis, and chronic obstructive pulmonary disease.

29. The method of claim 1, wherein the therapeutic agent is an antiinflammatory agent selected from the group consisting of a corticosteroid, cromolyn, and nedocromil.

30. The method of claim 1, wherein the compound is a therapeutic agent for treating ischemia, Parkinson's disease, schizophrenia, cancer, acquired immune deficiency syndrome (AIDS), infections of the central nervous system, epilepsy, multiple sclerosis, neurodegenerative disease, trauma, depression, Alzheimer's disease, migraine, pain, and a seizure disorder.

31. The method of claim 1, wherein the compound is selected from the group consisting of cyclosporin, insulin, a vasopressin, a leucine enkephalin, calcitonin, 5-fluorouracil, a salicylamide, a β-lactone, an ampicillin, a penicillin, a cephalosporin, β-lactamase inhibitor, a quinolone, a tetracycline, a macrolide, a gentamicin, acyclovir, ganciclovir, a trifluoropyridine, and pentamidine.

32. A composition comprising:
an effective amount of a biologically active agent;
a delivery-enhancing transporter having sufficient guanidino or amidino moieties to increase delivery of the biologically active agent across a biological barrier compared to the delivery of the biologically active agent in the absence of the transporter; and
a pharmaceutically acceptable carrier.

33. The composition of claim 32, wherein the biologically active agent is selected from the group consisting of antiviral agents, antibacterial agents, antifungal agents, antiproliferative agents, immunosuppressive agents, vitamins, analgesic agents and hormones.

34. The composition of claim 33, wherein the biologically active agent is an antiviral agent selected from the group consisting of acyclovir, famciclovir, ganciclovir, foscarnet, idoxuridine, sorivudine, trifluridine, valacyclovir, cidofovir, didanosine, stavudine, zalcitabine, zidovudine, ribavirin and rimantatine.

35. The composition of claim 32, wherein the biologically active agent is an antibacterial agent selected from the group consisting of nafcillin, oxacillin, penicillin, amoxacillin, ampicillin, cefotaxime, ceftriaxone, rifampin, minocycline, ciprofloxacin, norfloxacin, erythromycin and vancomycin.

36. The composition of claim 32, wherein the biologically active agent is an antifungal agent selected from the group consisting of amphotericin, itraconazole, ketoconazole, miconazole, nystatin, clotrimazole, fluconazole, ciclopirox, econazole, naftifine, terbinafine and griseofulvin.

37. The composition of claim 32, wherein the biologically active agent is an antineoplastic agent selected from the group consisting of pentostatin, 6-mercaptopurine, 6-thioguanine, methotrexate, bleomycins, etoposide, teniposide, dactinomycin, daunorubicin, doxorubicin, mitoxantrone, hydroxyurea, 5-fluorouracil, cytarabine, fludarabine, mitomycin, cisplatin, procarbazine, dacarbazine, paclitaxel, colchicine, and the vinca alkaloids.

38. The composition of claim 32, wherein the biologically active agent is an immunosuppressive agent selected from the group consisting of methotrexate, azathioprine, fluorouracil, hydroxyurea, 6-thioguanine, chclophosphamide, mechloroethamine hydrochloride, carmustine, cyclosporine, taxol, tacrolimus, vinblastine, dapsone and sulfasalazine.

39. The composition of claim 32, wherein the biologically active agent is an analgesic agent selected from the group consisting of lidocaine, bupivacaine, novocaine, procaine, tetracaine, benzocaine, cocaine, mepivacaine, etidocaine, proparacaine ropivacaine and prilocaine.

40. The composition of claim 33, wherein the delivery enhancing transporter is a peptide having from about 6 to about 15 amino acids residues wherein from 6 to about 12 residues are selected from the group consisting of L-arginine, D-arginine, L-homoarginine and D-homoarginine.
